(19) United States
(12) Patent Application Publication
(10) Pub. No.: US 2010/0168609 A1
(43) Pub. Date: Jul. 1, 2010

(54) BIOPSY DEVICE FOR THE ENRICHMENT OF TISSUE, CELLS, OR ANALYTES

(75) Inventors: Ulrich Pison, Berlin (DE); Axel Schaefer, Berlin (DE)

Correspondence Address: JOYCE VON NATZMER;PEQUIGNOT + MYERS LLC, 200 Madison Avenue, Suite 1901, New York, NY, 10016, US

(73) Assignee: OGENO GMBH, Berlin (DE)

(21) Appl. No.: 12/530,945

(22) PCT Filed: Mar. 14, 2008
(86) PCT No.: PCT/EP2008/002416

§ 371 Date: Feb. 26, 2010

(30) Foreign Application Priority Data
Mar. 14, 2007 (DE) 10 2007 013 008.4

(51) Int. Cl. A61B 10/02

(52) U.S. Cl. 600/562

(57) ABSTRACT

The invention relates to a biopsy device for the in vivo enrichment of tissue, cells, or analytes, including drugs and therapeutic active substances, at the site of biopsy and for subsequent testing.

The present invention relates generally to medical instrumentation and more particularly to biopsy devices.

Biopsy devices in general are used to obtain samples of tissue, cells or analytes out of the body of animal or human for subsequent testing. In order to obtain a biopsy sample, the biopsy device must be inserted into the body to reach the appropriate compartment for sampling tissue, cells or analytes of interest. One major limitation of biopsy in general is that the obtained sample does not include the tissue, cell or analyte of interest. To increase the probability that the material of interest is sampled in an appropriate concentration to be detectable in subsequent testing multiple biopsies could be taken or the sample volume could be increased. The number of biopsies and increasing sampling volume, however, is limited in living animal and human.

To achieve the foregoing and other objects and in accordance with the purpose of the present invention as embodied and described herein, the present invention is directed to a functionalized biopsy device for the in vivo enrichment of tissue, cells, or analytes (including drugs and therapeutic active substances) at the site of biopsy. The probability that the material of interest is sampled in the appropriate concentration for subsequent testing is achieved by keeping the device in position inside the body for a period of time, but without increasing the number of biopsies or sample volume. The site of biopsy could be solid tissue, but is preferable a cavity of the body like the peritoneum, or the uterus or the gastro intestinal tract, most preferably the lumen of artery or vein, or the ureter, or vasa lymphatica, or ductus choledochus, or ductus pancreaticus, or the spinal canal.

The method of enrichment, generally, is based on affinity interactions between the material of interest and the surface of the device. The affinity interaction is based on specific receptor-ligand interaction, or unspecific adhesion, or on physical attraction between the device and the material of interest.

In one embodiment of the invention, the biopsy device is a modified spring wire or flexible plastic rod or a catheter or stent as related to the invention for the capturing of rare cells in living animal and human.

In another embodiment of the invention, the biopsy device (spring wire, flexible plastic rod, catheter, or stent) is functionalized for capturing of rare analytes, including biomarkers, drugs and substances like radioactive tracers intended for diagnoses or therapy.

In yet another embodiment of the invention, the biopsy device (spring wire, flexible plastic rod, catheter, or stent) is functionalized for physical attraction of analytes or particles using physical characteristics of the analyte or particle.

In yet another embodiment the biopsy device (spring wire, flexible plastic rod, catheter, or stent) is functionalized for capturing of cells like embryonic or adult stem cells inside the body using collagen pads.

Biopsy devices to obtain samples of tissue, cells or analytes out of the body of animal or human for subsequent testing are described in prior art (see for example US Patent US2002026188). Some of these systems are rather complex and are often assembled using several elements, e.g., comprising microsystems for investigation of a substrate and/or for delivery of active agents in a substrate, a flexible rod to one end of which the microsystem is attached, and the other end of which is intended for the control of said microsystem (see US Patent US2003049679). Others contain catheters with optical fiber sensors (U.S. Pat. No. 7,329,223). Again others employ nanosystems as arrays of biological molecules placed in given positions on a surface providing a diagnostic biosensor (see WO 2006131400).

Prior art disclose enrichment techniques of cells and analytes after biopsy (see for example W02006108087) but not the enrichment of tissue, cells, or analytes at the site of biopsy in vivo for subsequent testing. We have used nanosystems as arrays of biological molecules placed in given positions on a surface providing a diagnostic biosensor before (see WO 2006131400). Using the approach disclosed herein a much simpler but more efficient technique is used for the capturing of rare cells and analytes inside living systems.

The present invention is directed generally to a method and device for the in vivo enrichment of rare tissue, cells or analytes at the site of biopsy for subsequent testing.

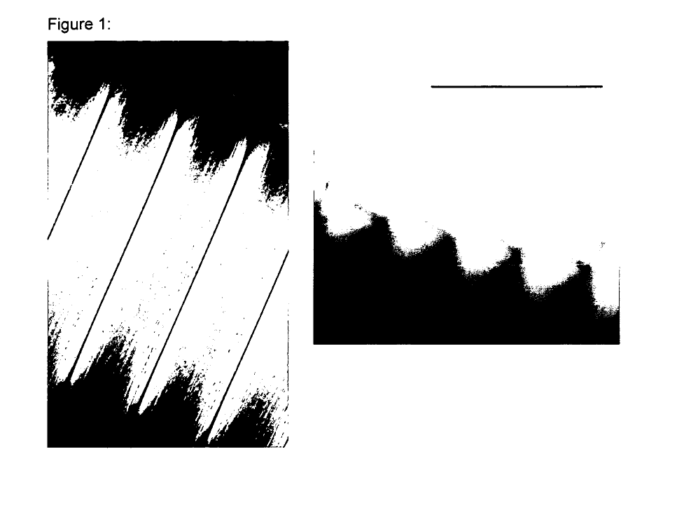

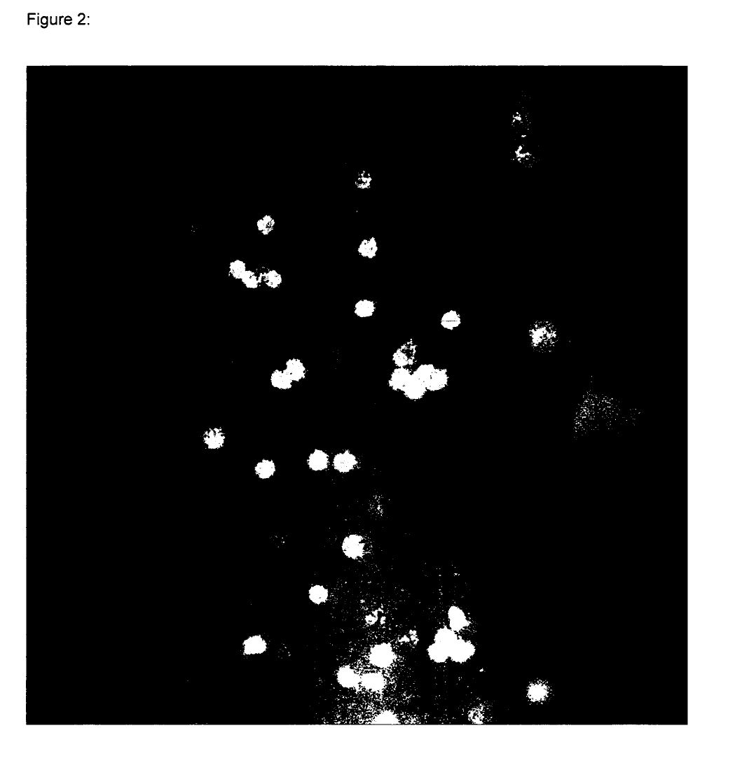

In a first embodiment of the invention, the biopsy device is a modified spring wire or flexible plastic rod or catheter or stent as related to the invention for the immunocapturing of rare cells (the targets) in living animal and human. In this embodiment the tip of the spring wire (1 to 2 cm in length) is electroplated with gold or metals preferable from group 10 or 11 of the periodic table of the elements (see FIG. 1). After activation of the metal surface specific ligands like antibodies, parts thereof, or other sequences that contain the binding structure for the corresponding membrane receptor of the cell of interest are covalently bound through thiol bonds and using acid termination. Alternatively the tip of the spring wire or the flexible plastic rod or catheter or stent could be modified with metals using evaporation techniques, ceramic techniques or cementation techniques for the subsequent binding of the ligand. In addition, the tip of the spring wire or flexible plastic rod or catheter or stent could be used directly for binding of the target, if a corresponding binding structure is not necessary such as in case of unspecific adhesion. Furthermore, the tip of the spring wire or flexible plastic rod or catheter or stent could be also used directly (without ligand) for binding of the target, if a corresponding binding structure is imprinted unto the tip of the device.

The invention relates also to a biopsy device for the in vivo enrichment of tissue, cells, or analytes, including drugs and therapeutic active substances, at the site of biopsy for subsequent testing where the device or part of the device is electroplated with gold or metals preferably from group 10 or 11 of the periodic table of the elements. It was utterly surprising that the device or part of the device which is electroplated with gold or metals, preferably from group 10 or 11 of the periodic table of the elements is surprisingly better than the devices of the state of the art. With the biopsy devices of the invention, cells, analytes or tissue can be detected and extracted surprisingly well from a sample. As a special advantage of this preferred embodiment of the invention, biological molecules are detected in an especially safe way.

In another preferred embodiment of the invention, the device or part of the device is modified with metals preferably from group 10 or 11 of the periodic table of the elements using evaporation techniques. It was utterly surprising that use of such evaporation techniques leads to a biopsy device which is surprisingly better than the devices of the state of the art. As a special advantage of this preferred embodiment of the invention, the in vivo enrichment of the molecules is effected in an especially specific way.

In yet another preferred embodiment of the invention, the device or part of the device is modified with metals preferably from group 10 or 11 of the periodic table of the elements using ceramic techniques. Again, it was utterly surprising that use of such ceramic techniques leads to a biopsy device which is surprisingly better than the devices of the state of the art. As a special advantage of this preferred embodiment of the invention, the biopsy device leads to a minimum of collateral effects.

In yet another preferred embodiment of the invention, the device or part of the device is modified with metals preferable from group 10 or 11 of the periodic table of the elements using cementation techniques. Again, it was utterly surprising that use of such cementation techniques leads to a biopsy device which is surprisingly better than the devices of the state of the art. As a special advantage of this preferred embodiment of the invention, the molecules of interest are accumulated in an especially effective way.

In yet another preferred embodiment of the invention, the biopsy device of the invention has an outer form like a cylinder or a tube with a diameter ranging between 0.01 mm to 10 mm, preferably between 0.1 to 2 mm, most preferably between 0.25 mm to 0.8 mm. This preferred embodiment of the invention has shown special advantages for the in vivo enrichment of tissue cells or analytes.

In yet another preferred embodiment of the invention, the length of the biopsy device of the invention ranges from 0.5 mm to 300 mm, preferably from 10 mm to 100 mm, most preferably from 25 mm to 80 mm.

In yet another preferred embodiment of the invention, the dimensions or the core materials of the biopsy device of the invention are spring wires, flexible plastic rods, catheters, and/or stents. The dimensions and substrate materials are known to a person skilled in the art.

In yet another preferred embodiment of the invention, the surface of the device is further modified using anodic oxidation.

In yet another preferred embodiment of the invention, the device is decorated with a specific ligand selected from a group comprising: antibodies, parts thereof, or other sequences with high binding affinity to tissue, cells, or analytes of interest at the site of biopsy.

In yet another preferred embodiment of the invention, the ligand is coupled to the device using standard techniques known to a person skilled in the art. It was utterly surprising that the binding with a specific ligand leads to especially advantageous uses. Even if a person with average skill in the art may have assumed that the binding of specific ligands would lead to an improvement of the in vivo enrichment of tissue, cells or analytes, it was utterly surprising that the biopsy device of the invention would allow such a great number of advantageous uses.

In yet another preferred embodiment of the invention, the device could be placed inside the body of an animal or human for a defined period of time. The invention therefore also relates to the use of the biopsy device of the invention inside the body of an animal or a human for a defined period of time. This defined period of time may last for 1 to 120 minutes, preferably 10 to 60 minutes, most preferably 30 minutes. It was utterly surprising that these defined periods of time leads to an especially efficient in vivo enrichment of tissue, cells or analytes.

Preferred location places in vivo could be solid tissue, preferably a cavity of the body like the peritoneum or uterus, most preferable the lumen of artery or vein, or the ureter, or vasa lymphatica, or ductus choledochus, or ductus pancreaticus, or the spinal canal. It was especially surprising that the in vivo enrichment of tissue, cells or analytes can be carried out especially efficiently, quickly and safely.

In yet another preferred embodiment of the invention, the period of time in vivo is determined by the concentration of the material of interest and the affinity between the ligand and the material of interest.

In yet another preferred embodiment of the invention, the device is magnetic and thus could attract magnetic particles.

In yet another preferred embodiment of the invention, the surface of the device of the invention is further modified using anodic oxidation. This results in a surprisingly effective enrichment of the molecules in the sample.

The binding capacity for the ligand could be further improved using secondary surface modifications after plating including anodic oxidation and chemical activation.

Applications, using this embodiment of the invention, particularly relate to but are not limited to the following: enrichment of cancer cells, or micro-metastases; enrichment of foetal cells in maternal blood; identification of infectious diseases through the enrichment of viruses or fungi or parasites or elements thereof; enrichment of rare or abnormal cells that are indicative for a state of disease. An example of captured CD4+ cells is given in FIG. 2.

In yet another embodiment of the invention, the biopsy device (spring wire, flexible plastic rod, catheter, or stent) is functionalized for immunocapturing of rare analytes, including biomarkers, drugs and substances like radioactive tracers intended for diagnoses or therapy. An analyte could be an atom, molecule, group of molecules or compound of natural or synthetic origin (e.g., drug, hormone, enzyme, protein, peptide, protein complex, antigen, antibody, hapten, lectin, apoprotein, cofactor) sought to be detected or measured that is capable of binding specifically to at least one binding partner (e.g., drug, hormone, antigen, antibody, hapten, lectin, apoprotein, cofactor). Analytes vary in size. Merely by way of example, small molecule analytes may be, for instance, <0.1 nm. However, analytes may be larger than this, including for instance immunoglobulin analytes (such as IgG, which is about 8 nm in length and about 160,000 Daltons) or other protein complexes.

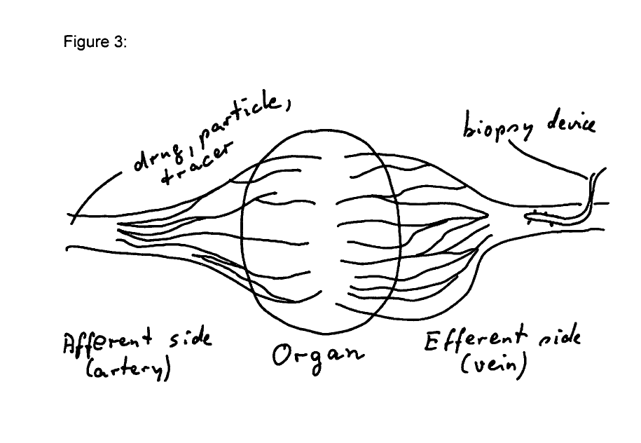

Of the several possible applications, using this embodiment of the invention particularly relate to but are not limited to the following: enrichment of biomarker at the site of biopsy for ex vivo testing; capturing of that part of drug used for targeted therapy and its subsequent elimination, that is spilled over after saturation of the target; capturing of radiotracer that persist in the circulation after first passing the target organ. FIG. 3 illustrates this concept.

In yet another embodiment of the invention, the biopsy device (spring wire, flexible plastic rod, catheter, or stent) is functionalized for physical attraction of analytes or particles using physical characteristics of the analyte or particle. In this regard, we have used the magnetic vector of the device made out of ferritic steel to capture magnetic particles inside the circulation. For this specific application the device is positioned in the efferent part of the vascular system that supplies an organ, whereas the particles are injected in the afferent part of the vascular system.

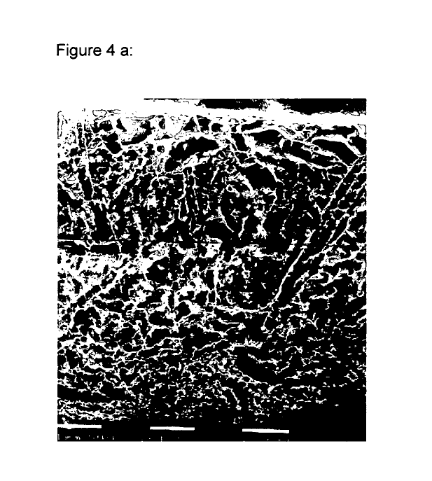

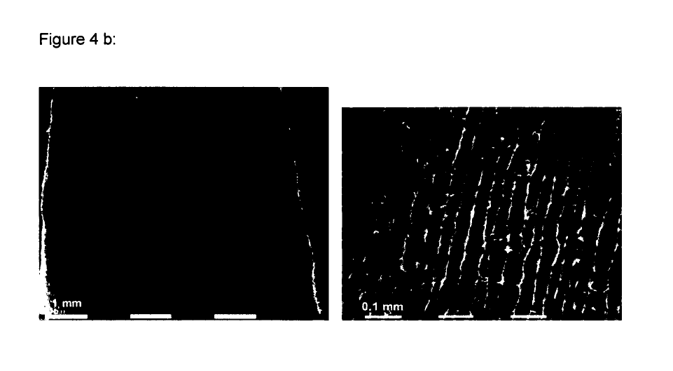

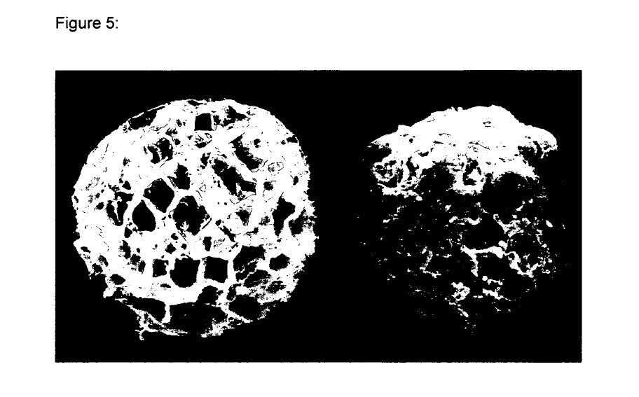

In yet another embodiment the biopsy device (spring wire, flexible plastic rod, catheter, or stent) is functionalized for capturing of cells like embryonic or adult stem cells inside the body. The tip of the device is functionalized using collagen pads. The collagen pads differ from commercially available pads (FIG. 4a) in the sense that there pores structure is homogenous (see FIG. 4b). Spherical collagen pads modifying the tip of the biopsy device are implanted into the peritoneum cavity for the enrichment of adult stem cells as depicted in FIG. 5.

It was utterly surprising that the advantageous characteristics of the device of the invention could be realised without nanotechnology and without nanostructures. It is therefore possible to check the produced devices with a magnifying glass or another magnifying device, instead of having to use a microscope as is necessary for devices which feature a nanostructure or which are produced using nanotechnology.

It was furthermore surprising that the ligands, for example antibodies, bind better to the device of the invention if the device does not feature nanostructures. For example, it is possible to coat blanks made from steel or plastic, either entirely or on their tip, preferably with gold using electroplating, ceramic, cementation and/or evaporation techniques. It is not necessary to treat the entire device or the tip of the device for example with nanolithography, so that the device features a nanostructure. Nanostructures or nanotechnologies in the context of this invention are preferably defined as those disclosed in the EP 1 811 302 A1.

Biopsy devices in general are used to obtain samples of tissue, cells or analytes out of the body of an animal or human for subsequent testing. The number of biopsies and increasing sampling volume, however, is limited in living animal and human. The new functionalized biopsy devise as disclosed herein allows the enrichment of the material of interest in vivo. Thus, the described biopsy devices lead to surprising results and is not obvious for the following reasons:
departure from the beaten track
a new perception of the problem
satisfaction of a long-felt need or want
hitherto all efforts of experts were in vain
the simplicity of a solution proves inventive action, especially if it replaces a more complex doctrine
the development of scientific technology moved in another direction
the achievement forwards the development
misconceptions about the solution of the according problem (prejudice)
technical progress, such as: improvement, increased performance, price-reduction, saving of time, material, work steps, costs or resources that are difficult to obtain, improved reliability, remedy of defects, improved quality, no maintenance, increased efficiency, better yield, augmentation of technical possibilities, provision of another (not necessarily better) product, opening of a second (not necessarily better) way, opening of a new field, first solution for a task, spare product, alternatives, possibility of rationalisation, automation or miniaturisation or enrichment of the pharmaceutical fund
special choice (if a certain possibility, the result of which was unforeseeable, is chosen among a great number of possibilities, that is a patentable lucky choice)
error in a citation
young field of technology
combined invention; a combination of a number of known elements, with a surprising effect
licensing
praise of experts and
commercial success

The figures show the following aspects:

FIG. 1: Spring wire before (left) and after electroplating with gold (right). The ruler in the right figure gives 0.05 mm.

FIG. 2: Captured CD4+ cells on electroplated spring wire that have been decorated with a monoclonal antiCD4 antibody. CD4+ cells were obtained from the peripheral circulation of men using the new biopsy device. Cells were visualized after biopsy using fluorescence microscopy.

FIG. 3: Drugs, particles, or tracers used for, e.g., targeted diagnoses or therapy are captured in the efferent part of the circulation system of an organ. After first pass through the organ most of the drug or particle or tracer will remain in the organ due to specific interaction or unspecific filtering.

Some of the drug or particle or tracer, however, is spilled over into the efferent part of the circulation. The biopsy device captures this part of the drug or particle or tracer thanks to its interaction with the analyte.

FIG. 4a: Commercially available collagen pads

FIG. 4b: The collagen pads with homogenous pores structure

FIG. 5: Spherical collagen pads (diameter: 0.6 mm, left without cells, right with

CD35+ cells) modifying the tip of the biopsy device for the enrichment of adult stem cells.

1. A biopsy device comprising said device and wherein the device or part of the device is electroplated with gold or metals preferably from group 10 or 11 of the periodic table of the elements, wherein the device is for in vivo enrichment of tissue, cells, or analytes, including drugs and therapeutic active substances, at the site of biopsy for subsequent testing.

2. A biopsy device according to claim 1, wherein the device or part of the device is modified with metals preferably from group 10 or 11 of the periodic table of elements using evaporation techniques.

3. A biopsy device according to claim 1, wherein the device or part of the device is modified with metals preferably from group 10 or 11 of the periodic table of elements using ceramic techniques.

4. A biopsy device according to claim 1, wherein the device or part of the device is modified with metals preferably from group 10 or 11 of the periodic table of elements using cementation techniques.

5. A biopsy device according to claims 1 to 4, wherein the surface is further modified using anodic oxidation.

6. A biopsy device according to claim 1, wherein the device or parts thereof are not treated with nanotechnology or do not feature nanostructures which are obtained by nanotechnological methods.

7. A biopsy device comprising a device and wherein the device or part of the device is modified with collagen pads and wherein the device is for in vivo enrichment of stem cells and others, including drugs and therapeutic active substances, at the site of biopsy for subsequent culturing or testing.

8. A biopsy device according to claim 1 or claim 7, wherein the device has an outer form of a cylinder or a tube with a diameter ranging from 0.01 mm to 10 mm, preferably from 0.1 to 2 mm, most preferably from 0.25 mm to 0.8 mm.

9. A biopsy device according to claim 1, wherein the device has a length between 0.5 mm to 300 mm, preferably between 10 mm to 100 mm, most preferably between 25 mm to 80 mm.

10. A biopsy device according to claim 1 or 7, wherein dimensions or core materials of the biopsy device are spring wires, flexible plastic rods, catheters, and/or stents.

11. (canceled)

12. A biopsy device according to claim 1, wherein the device is decorated with a specific ligand selected from a group consisting of antibodies, parts thereof, or sequences, which are not antibodies or parts thereof, having high binding affinity to tissue, cells, or analytes of interest located at the site of biopsy.

13. A biopsy device according to claim 1 with a surface that is further modified using imprint techniques to serve as a corresponding binding structure for a target.

14. A biopsy device according to claim 1 that is to be placed inside a body of an animal or human for a defined period of time, wherein the a location for placing the device in vivo is preferably solid tissue, more preferably a cavity of the body like the peritoneum or uterus or gastro intestinal tract, most preferably the lumen of artery or vein, or the ureter, or vasa lymphatica, or ductus choledochus, or ductus pancreaticus, or the spinal canal.

15. A biopsy device according to claim 14, wherein the period of time in vivo is determined by the a concentration of the a material of interest and the a binding affinity between the ligand and the material of interest.

16. A biopsy device according to claim 1 that is magnetic and thus could attracts magnetic particles.

17. A biopsy device according to claim 7, wherein the a period of time in vivo within which the device is placed inside a body of an animal or human is determined by a concentration of a material of interest and a binding affinity between the collagen pad and the material of interest.

18. A biopsy device according to claim 13, wherein a period of time in vivo within which the device is placed inside a body of an animal or human is determined by a concentration of the a material of interest and the a binding affinity between the imprinted structure of the a tip of the device and the material of interest.
